(19) United States
(12) Patent Application Publication
(10) Pub. No.: US 2008/0140198 A1
(43) Pub. Date: Jun. 12, 2008

(54) METHOD FOR ENHANCED FOLLICULAR TISSUE TRANSPLANTATION AND MANAGEMENT

(76) Inventor: Robin Unger, New York, NY (US)

Correspondence Address: EDWARD ETKIN, ESQ., Law Office of Edward Etkin, PC, 228 West End Avenue, Suite A, Brooklyn, NY, 11235, US

(21) Appl. No.: 11/952,076

(22) Filed: Dec. 6, 2007

Related U.S. Application Data
(60) Provisional application No. 60/873,329, filed on Dec. 6, 2006.

(51) Int. Cl. A61F 2/10

(52) U.S. Cl. 623/15.11

(57) ABSTRACT

An method for enhanced follicular tissue transplantation and management is provided. Follicular tissue is first obtained from a donor during a dedicated harvesting procedure or as a by-product of another cosmetic surgery procedure, then cryopreserved and stored. When implantation is desired, the cryopreserved tissue is thawed, and the tissue that survived the cryopreservation and thawing processes is implanted in the recipient. In another aspect of the invention, the harvesting process is conducted when the quality of donor follicular tissue is as optimal as possible, and is stored in cryopreserved form until the donor requires implantation thereof, such that the newly implanted thawed follicular tissue is of higher quality than the recipient's current follicular tissue. In yet another aspect of the present invention, a method for managing the business aspects of the transplantation process, including harvested follicular tissue grading, cataloguing and banking is provided.

### CROSS REFERENCE TO RELATED APPLICATION

The present patent application claims priority from the commonly assigned U.S. Provisional Patent Application Ser. No. 60/873,329, entitled “METHOD FOR ENHANCED FOLLICULAR TISSUE TRANSPLANTATION AND MANAGEMENT” filed Dec. 6, 2006.

### FIELD OF THE INVENTION

The present invention relates generally to an enhanced method of transplantation of follicular tissue, and more particularly to a method for harvesting follicular tissue from a donor during an opportune time, cryopreserving and storing the tissue, and then advantageously implanting it at a later point in the recipient, who may be the original donor, or who may be another person.

### BACKGROUND OF THE INVENTION

Procedures for transplantation of follicular tissue (otherwise known as “hair transplantation”) are well known in the art of cosmetic surgery. Typically such procedures involve the steps of harvesting viable follicular tissue (e.g., in form of blocks, strips, or by follicular unit extraction), from suitable hair bearing regions of the scalp, or from another region of the body, and then, as part of the same surgical procedure, implanting the harvested follicular tissue in one or more other regions of the scalp (or the body). However, these commonly known procedures suffer from a number of disadvantages:

1) performing both harvesting and implantation procedures during the same surgery session can be difficult and uncomfortable for the patient, and as a result several separate transplantation procedures are typically necessary to achieve the desired result; and

2) by the time most patients develop a need for hair transplantation, their follicular tissue has already deteriorated from its optimal condition (because follicular tissue deteriorates as a person grows older), and thus not only is there a limitation on the quantity of viable follicular tissue suitable for transplantation, but the quality of the tissue is likely far from optimal.

In addition, during many cosmetic surgery procedures (and especially in facial cosmetic surgery), viable follicular tissue is excised as part of the procedure and then discarded.

It would thus be desirable to provide a method for enhanced follicular tissue transplantation that addresses the drawbacks of conventional hair transplantation procedures. It would also be desirable to provide a method for enhanced follicular tissue transplantation that enables future utilization of follicular tissue produced as a by-product of cosmetic surgery procedures. It would also be desirable to provide a method for managing the enhanced transplantation between the same, or different individuals.

### BRIEF DESCRIPTION OF THE DRAWINGS

In the drawings, wherein like reference characters denote corresponding or similar elements throughout the various figures:

FIGS. 1A-1B are a schematic block diagram of an exemplary first embodiment of a process for enhanced follicular tissue transplantation;

FIG. 2 is a schematic block diagram of an exemplary embodiment of sub-processes of the inventive process of FIG. 1, for obtaining follicular tissue; and

FIG. 3 is a schematic block diagram of an exemplary embodiment of a process for managing follicular tissue transplantation processes of FIGS. 1A, 1B and 2 between multiple donors and recipients.

### SUMMARY OF THE INVENTION

The system and method of the present invention are directed to a process of enhanced follicular tissue transplantation and management. Follicular tissue is first obtained from a donor during a dedicated harvesting procedure or as a by-product of another cosmetic surgery procedure, then cryopreserved and stored. When implantation is desired, the cryopreserved tissue is thawed, and the tissue that survived the cryopreservation and thawing processes is implanted in the recipient. In another aspect of the invention, the harvesting process is conducted when the quality of donor follicular tissue is as optimal as possible, and is stored in cryopreserved form until the donor requires implantation thereof, such that the newly implanted thawed follicular tissue is of higher quality than the recipient's current follicular tissue. In yet another aspect of the present invention, a method for managing the business aspects of the transplantation process, including harvested follicular tissue grading, cataloguing and banking is provided.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### DESCRIPTION OF THE PREFERRED EMBODIMENTS

The present invention advantageously overcomes the drawbacks and disadvantages of previously known hair transplantation techniques and provides new advantages and benefits by virtue of its multi-stage approach.

In summary, in one exemplary embodiment of the invention, follicular tissue is first obtained from a donor during a dedicated harvesting procedure or as a by-product of another cosmetic surgery procedure utilizing any desired harvesting technique, the tissue is then cryopreserved and stored. When implantation is desired at a later time (for example when the donor grows older and experiences hair loss), the cryopreserved tissue is thawed, and the tissue that survived the cryopreservation and thawing processes is implanted in the recipient. Thus, it may be advantageous (but not essential) to conduct the initial harvesting process when the quality of donor follicular tissue is as optimal as possible, such that the tissue thawed and implanted at a future point is of higher quality than the recipient's then-current follicular tissue. In another embodiment of the present invention, a method for managing the business aspects of the transplantation process, including harvested follicular tissue grading, cataloguing and banking us provided.

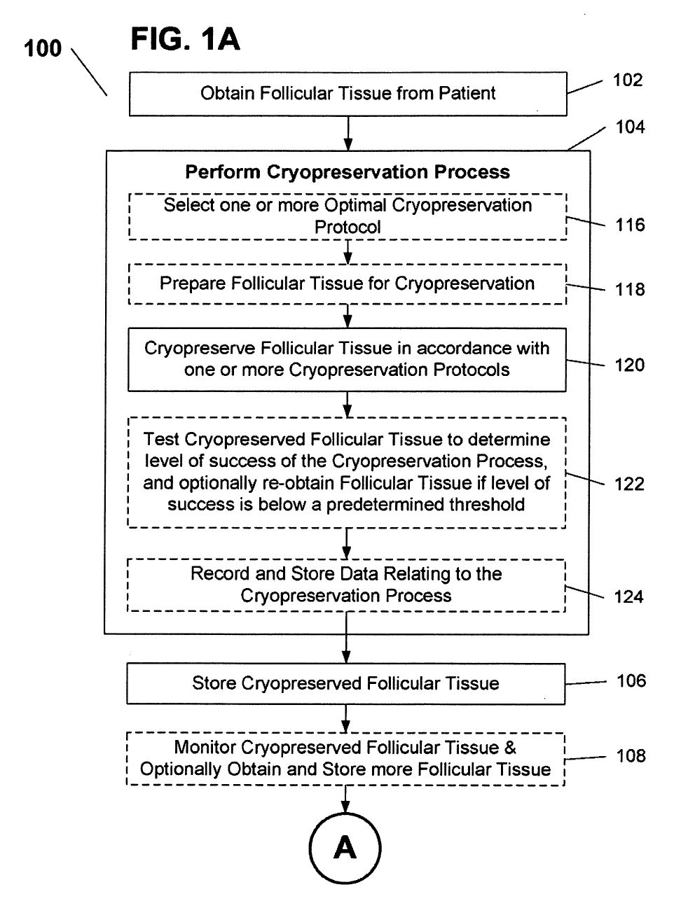

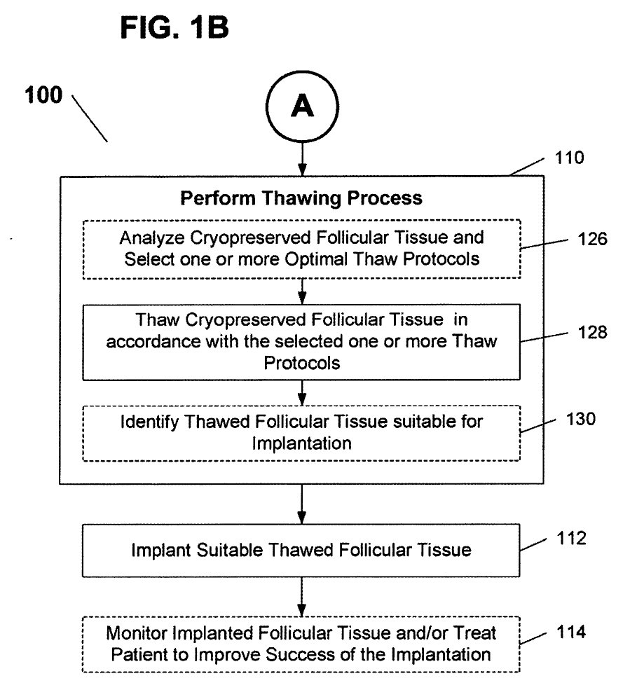

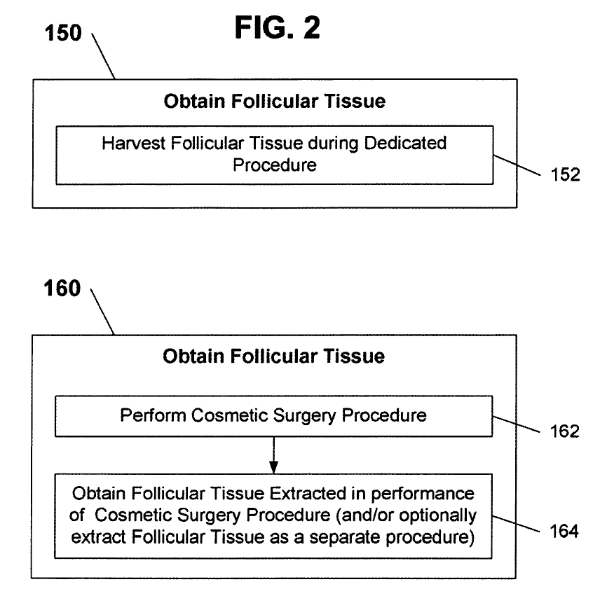

Referring now to FIGS. 1A, 1B and 2, an exemplary embodiment of the method for enhanced follicular tissue transplantation is shown as a process 100. At a step 102 the follicular tissue is obtained (for example as shown in obtaining sub-processes 150 or 160 in FIG. 2), cryopreserved at a sub-process step 104 (which includes step 120 and optional steps 116, 118, 122 and 124), and then stored at a step 106, and optionally monitored at an optional step 108 (whereupon additional follicular tissue may be obtained as necessary or desired). After a period of time (which may range from days to years), when implantation of the follicular tissue is desired, a thawing process 110 (which includes step 128 and optional steps 126 and 130) is performed, and the thawed follicular tissue is implanted at a step 112. Thereafter at an optional step 114, the implanted tissue may be monitored and the patient may be treated (for example by providing anti-tissue rejection medication) to improve success of the implantation.

As shown in FIG. 1A, optional steps, such as selecting one or more optimal cryopreservation protocols (optional step 116), preparing the follicular tissue for cryopreservation (optional step 118), and verifying the viability of cryopreserved tissue (optional step 122) may be performed as a matter of design choice or necessity. It should also be noted that the present invention contemplates that certain steps (e.g., steps 102 to 106) may be repeatedly performed over a period of time to accumulate a desired quantity of stored cryopreserved follicular tissue.

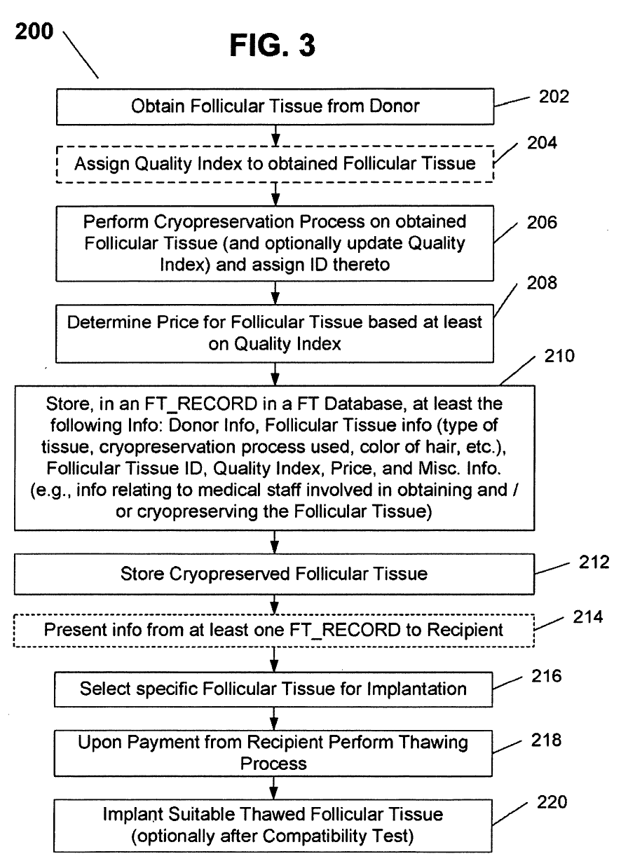

Referring now to FIG. 3, a method for managing multiple enhanced transplantation processes is shown as a process 200. The process 200 utilizes certain aspects of the process 100 of FIG. 1 (such as steps 202, 206, 212, 218 and 220), but also includes steps for grading the quality of obtained follicular tissue (optional step 204), pricing the stored tissue (step 208), creating and maintaining records (step 210) and offering one or more choices of tissue to the recipient (step 214). It should be noted that in accordance with the present invention, the donor and recipient may be the same individual, or they may be different individuals. If transplantation between different individuals is desired, the optional tissue compatibility test at the step 220 is very advisable prior to implantation.

Thus, while there have been shown and described and pointed out fundamental novel features of the invention as applied to preferred embodiments thereof, it will be understood that various omissions and substitutions and changes in the form and details of the devices and methods illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention.

1. A method for transplanting follicular tissue from a donor to a recipient comprising the steps of:
(a) harvesting a predefined quantity of follicular tissue from the donor;
(b) cryopreserving said predefined quantity of follicular tissue;
(c) storing said cryopreserved follicular tissue;
(d) selectively thawing said stored cryopreserved follicular tissue; and
(e) implanting said thawed cryopreserved follicular tissue in the recipient.
